(19) United States
(12) Patent Application Publication
(10) Pub. No.: US 2022/0291245 A1
(43) Pub. Date: Sep. 15, 2022

(54) SPECIFIC THC DETECTION DEVICE

(71) Applicant: Veriteque USA, Inc., Carson City, NV (US)

(72) Inventor: Christian Loane, Carson City, NV (US)

(21) Appl. No.: 17/770,625

(22) PCT Filed: Oct. 21, 2020
(86) PCT No.: PCT/US2020/056655

§ 371 Date: Apr. 21, 2022

Related U.S. Application Data
(60) Provisional application No. 63/000,338, filed on Mar. 26, 2020.
(60) Provisional application No. 62/923,859, filed on Oct. 21, 2019.

(51) Int. Cl. G01N 33/94; B01L 3/00; G01N 33/52

(52) U.S. Cl. CPC G01N 33/948; B01L 3/5023; G01N 33/521; B01L 2300/069; B01L 2200/16; B01L 2300/1805

(57) ABSTRACT

A portable detection kit for identifying the presence of cannabinoids is provided and includes a colorimetric dye, a catalytic reagent, a solvent, a surfactant, and a delivery device containing a solvent or solvent mixture including the solvent. The delivery device is configured to deliver a portion of the solvent or solvent mixture to a target residue to form a sample residue. The colorimetric dye is configured to undergo a chemical reaction when the sample residue contains a cannabinoid. The chemical reaction is configured to produce a visible color change that corresponds to a predetermined cannabinoid of a plurality of cannabinoids.

### CROSS-REFERENCE TO RELATED APPLICATIONS

This application claims the benefit of U.S. Provisional Patent Application No. 62/923,859, filed on Oct. 21, 2020 and entitled “Specific THC Detection Device,” and U.S. Provisional Patent Application No. 63/000,338, filed on Mar. 26, 2020, entitled “Specific THC Detection Device,” the entirety of each of which is incorporated by reference.

### FIELD

Embodiments of the present disclosure relate to a portable test kit capable of identifying and differentiating psychoactive tetrahydrocannabinol (THC) from other cannabinoid compounds including but not limited to cannabinol (CBN) and cannabidiol (CBD), based on simple rapid presumptive colorimetric color change. A process to cheaply mass produce said portable test kit and achieve long term commercial shelf life in the range of 1 to 5 years and a corresponding method to use the portable test kit are also provided.

### SUMMARY

Cannabis preparations, derived from the hemp plant, Cannabis sativa L., have been used by man for their euphoric effects for over four thousand years [1] and represent the most widely used of all drugs [2]. In 2018, data showed that 271 million people aged 15 — 64 had used drugs at least once in the previous year, of these 188 million were cannabis users and Cannabis attributed to 50% of all narcotics seized globally [2].

Cannabis contains over about 400 different chemical compounds. During smoking, more than about 2000 compounds may be produced by pyrolysis. The Cannabis plant produces natural product turpenes known as Cannabinoids which are the active chemicals used recreationally and medically throughout the world for various applications. Some Cannabinoids are considered psychoactive (e.g. THC, CBN), while other not (e.g. CBD). Over 100 Cannabinoids have currently been identified [3]. THC exists in several isomeric forms, including Δ10, Δ9, Δ8, Δ7, and Δ6-THC [6]. Technically only Δ49-THC is “illegal”. An example of the distribution of Δ9-THC in Cannabis plants and products is as follows: 10-12% in pistillate flowers, 1-2% in leaves, 0.1-0.3% in stalks, <0.03% in the roots [5]. The THC content of the different cannabis products (herb, resin and oil) is the result of the ratio of the different plant parts used in their production [5].

After being banned for decades, hemp cultivation in the USA has recently been reintroduced. The legal status of CBD in the USA is complicated, because many individual states have introduced their own medicinal or even recreational cannabis laws, while the Federal Government does not accept any consumption of cannabis [9]. In the USA, Germany and the UK, CBD is technically classified as a new medicine, requiring manufacturers to meet strict safety, quality, and effectiveness standards. In the Europe it is possible to cultivate certain cannabis varieties, which must be registered with EU's Common Catalogue of Varieties of Agricultural Plant Species. The THC content must not exceed 0.2%, in the USA and Canada 0.3%, while Switzerland THC content may go as high as 1% of the dried flowers of the plant, by weight [9]. The USA limit of 0.3% is currently being debated and is likely to change.

Many methods for the identification of cannabinoids currently exist, including but not limited to technical theory embodied in patent and scientific literature, laboratory procedures requiring highly sophisticated electronic equipment, and classical laboratory procedures all of which have limited or no field application, by way of example some of these are described below. Chemical analysis of Cannabis natural products has occurred for hundreds of years. Current methods of analysis, specifically geared towards identification of “illegal” Cannabis products include, but are not limited to, Presumptive tests, both Colorimetric and Immunoassay tests, Thin-layer chromatography (TLC), Ion mobility spectrometry (IMS), Gas chromatography (GC-FID) (GC-MS), Capillary chromatography, High-performance liquid chromatography (HPLC), Stable isotope ratio-mass spectrometry (IRMS) and DNA profiling [5].

Presumptive colorimetric techniques which have been employed historically, for identification of Cannabis extracts included, but are not limited to the following:

| Reagents | CBD | CBN | THC | CBC | CBD’ | CBDA |
|---|---|---|---|---|---|---|
| Ethanol/KOH [9] | V | — | — | — | V | V |
| Sodium acetate, 2,6- | | | | | | |
| dibromoquinone-4-chloride | Y | V | G | V | Y | Y |
| [9] | | | | | | |
| Ammoniacal silver [9] | G | — | — | — | G | G |
| Cinnamaldehyde/EtOH/ | R | BR | O | FP | R | R |
| HCl [9] | | | | | | |
| Vanillin/EtOH (Chloroform | BV | FB | B | PV | | |
| alternative)/HCl [9] | | | | | | |
| Dimethylamino | PV | BV | BV | P | P | |
| benzaldehyde/EtOH | | | | | | |
| (Chloroform alternative)/ | | | | | | |
| HCl [9] | | | | | | |
| Hydroxybenzaldehyde/ | BV | FB | BV | FB | FB | |
| EtOH (Chloroform | | | | | | |
| alternative)/HCl [9] | | | | | | |
| Anisaldehyde/Acetic acid/ | BR-V | BR | V | V | BR-G | |
| Sulfuric acid with heating [9] | | | | | | |
| Cinnamaldehyde/Acetic | O-BR | BR | O | BR-V | O-YGR | |
| acid/Sulfuric Acid [9] | | | | | | |
| Cinnamaldehyde/Acetic | R-G- | O | YGR | G | R-YGR | |
| acid/Sulfuric Acid/Heat | GR | | | | | |
| [9] | | | | | | |
| Tetrazotised dianisidine [9] | R | V | RV | V | O | R |
| Tetrazotised tolidine [9] | O | V | R | P | O | R |
| Tetrazotised benzidine [9] | O | V | R | BR | Y | O |
| Fast Blue (BB) | O | CR | V | | | |
| Duquenous modifications | BG | BG | BG | BG | BG | BG |
| vanillin, salicylaldehyde, p- | | | | | | |
| dimethylaminobenzaldehyde, | | | | | | |
| nitrobenzaldehyde and | | | | | | |
| hydrochloric acid extraction | | | | | | |
| into petroleum/ether | | | | | | |
| solvents [11] (Chloroform | | | | | | |
| alternative) | | | | | | |
| Ferric chloride + water/ | No specific color differentiation | | | | | |
| alcohol | | | | | | |
| 3% Iodic acid (HIO3) in | | | | | | |
| H2SO4 conc. + HNO3 conc. | | | | | | |
| (2:1) | | | | | | |
| Iron ammonium sulfate/ | | | | | | |
| potassium thiocyanate/ | | | | | | |
| toluene | | | | | | |
| Sulfanillic acid/sodium | | | | | | |
| nitrite [13] | | | | | | |
| Metaldehyde/vanillin/ | | | | | | |
| EtOH (Chloroform | | | | | | |
| alternative)/HCl [14] | | | | | | |
| Diazo red [14] | O | | | | | |
| Black bs [14] | V | | | | | |
| Black btl [14] | V | | | | | |
| Black g [14] | M | | | | | |
| Black k [14] | V | | | | | |
| Blue b [14] | RV | | | | | |
| Blue rr [14] | P | | | | | |
| Blue v [14] | O | | | | | |
| Blue vb [14] | — | | | | | |
| Blue vrt [14] | O | | | | | |
| Bordeaux bd [14] | OP | | | | | |
| Brown v [14] | RBR | | | | | |
| Corinth lb [14] | O | | | | | |
| Dark blue r [14] | V | | | | | |
| Garnet bgc [14] | GAR | | | | | |
| Garnet gc [14] | RO | | | | | |
| Grey g [14] | V | | | | | |
| Navy blue 3ra [14] | M | | | | | |
| Olive br [14] | OL | | | | | |
| Orange gc [14] | Y | | | | | |
| Orange ggd [14] | Y | | | | | |
| Orange gr [14] | YO | | | | | |
| Orange r [14] | Y | | | | | |
| Orange rd [14] | Y | | | | | |
| Red al [14] | OP | | | | | |
| Red b [14] | O | | | | | |
| Red frn [14] | YO | | | | | |
| Red itr [14] | O | | | | | |
| Red tr [14] | O | | | | | |
| Red 3gl [14] | O | | | | | |
| Scarlet r [14] | YO | | | | | |
| Violet b [14] | P | | | | | |
| Violet f [14] | OP | | | | | |
| Violet lb [14] | O | | | | | |

(KEY: FB—faint blue, BR—brown, R—red, O—Orange, Y—yellow, V—violet, P—pink, GR—green, DV—dark violet, CR—crimson, G—grey, M—Mauve, GAR—Garnet, OL—Olive)

All the above reagents are highly non-specific and give similar color changes to many other classes of narcotics. False positive results and the minimization of these is extremely important in regards to the use and reliance of presumptive test devices within the legal system [12]. Although these reagents are reported to give different colors to different Cannibinoids, extracted from the Cannabis plant, this visual differentiation is only possible when coupled with chromatographic separation of each individual compound, using high performance thin layer chromatographic separation of a Cannabis plant oil extracts. This methodology is simply not able to be constructed into small hand held kit format for field use. It requires the use of large volumes of hazardous liquid reagents, with many sequential steps and log periods of time, within a laboratory setting, for a successful and complete analysis. Many independent validation reports cite poor color differentiation, complicated methodology, and poor to no suitability of reagents systems to field use in kit form [10][12][14].

Laboratory bench mounted thin layer chromatography units are commercially available [15] however these devices are far from suited to mobile field deployment and requires many individual steps, large volumes of hazardous solvents and at least 30 minutes for a single complete analysis.

International Patent Publication WO 1999/054739 claims a wet chemical process for identification of Cannabinoids. The methodology discusses dissolution of reagents selected from fast black K, Fast blue B, 2,6-dibromoquinone-chlorimide, 2,6-dichloroquinone-chloroimide, vanillin, salicylaldehyde, formaldehyde, acetaldehyde, p-Dimethylaminobenzaldehyde, p-diethylaminobenzaldehyde, ferric chloride, 4-aminophenol or potassium hexacyanoferrate, in combination with alkali metal hydroxide or an alkali metal carbonate and/or an optionally substituted ammonium or alkali metal salt of an organic acid, creating a reaction solution in a primary, secondary and/or tertiary C1-C10 alcohols. The disclosure does not provide a specific test for THC, only discusses laboratory wet chemical process, and describes the requirement for non-corrosive vials and mixing chambers. The disclosure fails to provide a portable kit based format for the selective identification of THC.

US Patent Publication 2017/0234897 claims a method of manufacture of a cannabinoid quantification test strip impregnated with at least one cannabinoid-sensitive visualization reagent, wherein the visualization reagent comprises (A) potassium hydroxide, (B) modified Ghamrawy reagent consists of p-dimethylaminobenzaldehyde, combined with concentrated sulfuric or hydrochloric acid or p-toluenesulfonic acid and (C) Diazonium salts, Fast Blue B, Fast Blue BB, Fast Red B, Fast Red GG, Fast Orange GR, Fast Corinth V, Fast Garnet GC, Fast Red AV, and Fast Bordeaux GP. The disclosed method of manufacture requires dissolution of visualisation reagent in a solvent (e.g. water, methanol, ethanol, isopropanol, petroleum ether, methyl ethyl ketone, acetone, dimethylchloride, hexane), dip and dry the porous paper strip and place strip in air tight package, for later use. The disclosed method of use requires, contact of test strip with a test compound, where the test compound has previously been dissolved in a solvent. Dissolution of test compound requires the solid compound to be collected and transferred to a vial or similar, next addition of the extraction/dissolution solvent (i.e. methanol or propanol) to the solid sample, shaking for 10 s., the resulting extract solution to be transferred to an Eppendorf tube. A test strip is then dipped into the said Eppendorf extraction solution, the wet strip must be flick to remove excess solution and then air dried for 10 minutes to allow color formation.

The above disclosure has several points of concern regarding its claim to produce a kit for cannabinoid detection:
Point (A) above, the use of visualization reagent KOH. This is a hazardous caustic, corrosive reagent, which is extremely hygroscopic (i.e. absorbs atmospheric moisture). These characteristics, make this reagent, virtually impossible to (a) absorb into any porous/paper/synthetic fiber material (i.e. paper strip) as it cannot be dried (extremely hygroscopic) and will almost certainly react and destroy the material which it has been loaded into. In short you cannot produce a packaged paper strip with this reagent as it will have reacted and destroyed said paper strip within 48 hrs.
The KOH (above) is the only visualization reagent actually listed in the claims. All other visualization reagents are mentioned only in the detailed description. And the disclosure does not explain how the KOH will actually be put into the paper strip.
All visualization reagents disclosed, in any combination with the additional solid (toluenesulfonic acid) and liquid reagents (sulfuric and hydrochloric acids) and solvents (water, methanol, ethanol, isopropanol, petroleum ether, methyl ethyl ketone, acetone, dimethylchloride, hexane) will not provide any color differentiation between THC and other cannabinoids. They will give similar color development for many cross reacting drugs of abuse and household items (e.g. tea leaves, guaifenesin, nutmeg, cinnamon, curry leaves).
Specifically the combination kit composed of toluene sulfonic acid and aldehyde (DMAB) with solvents (water, methanol, ethanol, isopropanol, petroleum ether, methyl ethyl ketone, acetone, dimethylchloride, hexane) as disclosed in any combination will give no color differentiation between any individual cannabinoids. This combination kit gives slow development (30 sec.) of yellow to faint yellow green changing to pinks and red over 15 min., and gives similar color development with many cross reacting drugs of abuse and household ingredients.
The disclosed kit in its entirety does not achieve any form of THC color differentiation, is not compatible with true low cost mass production, does not disclose long term shelf life viability of all components, fails to identify correct “air tight packaging”, is not hand mobile and field deployable, contains large volumes of hazardous liquids, cannot be air-freighted without additional packaging and cost, and is better suited to a laboratory environment as it requires up to 12 individual steps and 15 minutes for complete single analysis.

U.S. Pat. No. 4,816,415 describes a means of filtering bodily fluids through a fibrous matrix which has an aryl carboxylic acid, specifically triphenylcarboxylic acid, pre-adsorbed into said matrix which has an affinity for, and binds to, cannabinoids. A secondary color change reagent solution is then poured over said matrix to afford a presumptive color change. The disclosure, which is specifically for and is designed bodily fluid analysis, fails to provide any form of cannabinoid differentiation and requires large volumes liquid reagents and so fails to meet the requirements of the current patent application.

U.S. Pat. No. 3,715,189 describes a plunger device, similar to a coffee plunger, to extract cannabinoids from materials. It utilizes hazardous solvents such as chloroform, and colorimetric dyes described for presumptive identification do not provide any form of cannabinoid differentiation. This disclosure fails to meet any of the requirement of the current patent application.

US Patent Publication No. 2015/0017732 discloses the use of a Dragendorf reagent for the detection of cannabinoids and synthetic cannabinoids. This is highly unusual and difficult to understand how this would work, given the Dragendorf reagent is specifically designed for identification of narcotic species containing highly reactive Nitrogen atoms, specifically in Alkaloids (e.g. Heroin) and Amines in general (e.g., Amphetamines). The cannabinoid group of chemicals contain no such reactive nitrogen groups. In any event the Dragendorf reagent does not provide differentiation between Nitrogeneous moieties.

US Patent Publication No. 2007/0077660 discloses a method for preparing cannabis samples for thin layer chromatography (TLC) analysis, requires large volumes of extracting solvents e.g. chloroform and 1,2-dichloro ethane, uses hazardous Sulfuric acid, requires heating elements, and utilises a range of non-specific color change reagents, including but not limited to: ceric ammonium molybdite, Iodine, UV light, dapsone, aniline, p-chloroanilin, p-toluidin, sulfadiazin, o-aminobenzoic acid, HMBT, cupric salts, ninhydrin, molybdenum blue reagent, vanillin, potassium permanganate and fluorescent dyes (e.g., primulin). The described device is non-field deployable, expensive to produce, and requires many steps and hazardous reagents for analysis. It fails to meet the requirements of the current application.

U.S. Pat. No. 4,771,005 discloses a method of utilizing diazonium salts under alkaline conditions combined together with hazardous solvents (e.g. dichloromethane, chloroform methanol, tetrahydrofuran, acetone and nitrobenzene), pre-packaged in ampoules or cartridges or cans. Most of the reagents described within this disclosure are either banned from current day manufacturing process, or require extremely expensive packing materials and costly hazardous material shipping licenses. This disclosure fails to achieve the requirements of the current application.

EP 18252621, U.S. Pat. No. 5,817,766, JP 2005-506520, and US 2019/0185946 all described variations on Immunoassay Antibody development and electronic detection of cannabinoid compounds.

International Patent Publication No. WO 1988/009496 describes a system for detecting cannabinoids in urine, which utilizes filters and azo dyes for cannabinoid detection. The system fails to differentiate between individual cannabinoids, is specifically for body fluid analysis. Ultimately the disclosure fails to meet the requirements of the current application.

U.S. Pat. No. 9,726,684 describes a Xanthene based fluorophore which fluoresces in the presence of cannabinoid species in the breath of subjects. This devices requires significant electronic instrumentation. It fails to meet the requirements of embodiments of the current application.

The disclosures highlighted above differ greatly in modality, construction, end use, to that of the kit described in the current application. Those described above are either for biological fluid analysis, based on immunoassay antibody detection techniques, require electronics, utilize large volumes of hazardous solvent and reagents, are mostly suited to laboratory use, and all have multiple steps and require large amounts of time to do a single complete analysis without achieving the differentiation of THC from other cannabinoids.

Accordingly, embodiments of the present disclosure describe a cheap, mass producible, highly portable, paper strip and swab device which requires little training and produces a visual presumptive colorimetric indication for THC, differentiates it, based on color, from CBD, CBN and other cannabinoids.

The preceding summary of patent references and public domain literature and disclosures are provided only as a point of reference in the difference(s) between laboratory equipment and procedures and true single step, cheap mass producible, field deployable presumptive colorimetric test kit for the detection of THC and broader cannabinoids, as disclosed herein.

An objective of the disclosed embodiments is to design a low cost, mass producible, field deployable, presumptive spot test kit which will facilitate identification of THC, differentiating it from other cannabinoids including but not limited to CBD and CBN, within suspect solid, gel, or liquid residues, while minimizing operator exposure.

It is a further objective of the disclosed embodiments to design both a presumptive kit, constructed of simple paper and an absorbent material (e.g., cotton swab products or wipes), with all color change reagents supplied in this format, constructed of non-hazardous materials and components.

Still a further objective of the disclosed embodiment is to design a portable presumptive test kit, which has true low cost, mass manufacture capability, in the order of millions of units per annum, while achieving commercial kit shelf life, in the order of several years and a reduced false detection rate.

Embodiments of the disclosure also provide a method of kit manufacture and use.

In an embodiment, a portable detection kit for identifying the presence of cannabinoids is provided and includes a colorimetric dye, a catalytic reagent, a solvent, a surfactant, and a delivery device containing a solvent or solvent mixture including the solvent. The delivery device is configured to deliver a portion of the solvent or solvent mixture to a target residue to form a sample residue. The colorimetric dye is configured to undergo a chemical reaction when the sample residue contains a cannabinoid. The chemical reaction is configured to produce a visible color change that corresponds to a predetermined cannabinoid of a plurality of cannabinoids.

In an embodiment, the plurality of cannabinoids includes one or more of tetrahydrocannabinol (THC), cannabidiol (CBD), or cannabinol (CBN).

In an embodiment, the delivery device includes an absorbent material.

In an embodiment, the delivery device is a cotton swab that absorbs the solvent or solvent mixture, a pop or snap cotton swab that stores the solvent or solvent mixture in a shaft, or a wipe that absorbs the solvent or solvent mixture.

In an embodiment, the colorimetric dye is received by a solid support carrier and the absorbent material contains the solvent mixture including the catalytic reagent and the surfactant. The solid support carrier and the absorbent material are each enclosed and separated from each other in respective containers.

In an embodiment, the solid support carrier is a paper card, a paper sheet, a synthetic paper, or Whatman filter paper.

In an embodiment, the solvent mixture further includes the colorimetric dye, the catalytic reagent, and the surfactant.

In an embodiment, the colorimetric dye is vanillin, the catalytic reagent is p-toluene sulfonic acid, and the surfactant is fumed silica.

In an embodiment, the composition of the dry mixture is: 77% w/w Vanillin, 18% w/w p-Toluene Sulfonic Acid, and 5% w/w fumed silica.

In an embodiment, the colorimetric dye, the catalytic reagent, and the surfactant are in the form of a dry mixture of powders of the colorimetric dye, the catalytic reagent, and the surfactant. The dry mixture and the absorbent material are each enclosed and separated from each other within respective containers.

In an embodiment, the delivery device is a non-absorbent container enclosing a solvent mixture including the colorimetric dye, the catalytic reagent, the surfactant, and the solvent.

In an embodiment, the non-absorbent container is a syringe, a spray can, a pump spray bottle, a breakable ampoule, a blister pack, or a dropper bottle.

In an embodiment, the kit further includes a heating device. The heating device is configured to produce heat sufficient to heat the residue in the form of a plant residue to a temperature greater than or equal to 100° C. for about 10 to about 60 seconds, thereby catalyzing conversion of cannabinoid species within the plant residue to psychoactive THC.

In an embodiment, the colorimetric dye is configured to undergo chemical reaction with the at least one cannabinoid in the form of liquids, gels or solid powders that are pure or admixed with cutting agents.

In an embodiment, the colorimetric dye is an aldehyde.

In an embodiment, the aldehyde is selected from the group consisting of Vanillin, DMAB, Metaldehyde, Anisaldehyde, Hydroxybenzaldehyde, Cinnamaldehyde, Saliscylaldehyde, or Nitrobenzaldehyde.

In an embodiment, the catalytic reagent increases the rate of appearance of the visible color change resulting from reaction between the colorimetric dye and the one or more predetermined cannabinoids.

In an embodiment, the catalytic reagent is at least one of a mineral acid or an organic acid in solid or liquid form.

In an embodiment, the catalytic reagent is selected from the group consisting of oxalic acid, citric acid, sodium bisulfate, or p-toluenesulfonic acid.

In an embodiment, the solvent is an alcohol.

In an embodiment, the solvent is selected from the group consisting of methanol, ethanol, isopropyl alcohol, butanol, or benzyl alcohol.

In an embodiment, the surfactant is selected from the group consisting of Anionic, Cationic, Zwitterionic, Non-ionic, C10-C20 Ethoxylates, Fatty acid esters, Amine oxides, Sulfoxides, Phosphine oxides, fumed silica, or plant-derived surfactants.

In an embodiment, the surfactant is Sodium Lauryl Sulfate.

In an embodiment, the visible color change includes:

a turquoise color formation when the residue contains tetrahydrocannabinol (THC);

a pink color formation when the residue contains cannabidiol (CBD) or cannabinol (CBN); and

no color change or a yellow color formation when the residue does not contain a cannabinoid.

In certain embodiments, a shade or lightness of the color produced by the color change can correspond to a concentration of a cannabinoid present in the sample residue.

In an embodiment, a method of preparing a portable detection kit for detecting the presence of cannabinoids is provided. The method includes storing a colorimetric dye, storing a catalytic reagent, storing a surfactant, and storing a solvent or solvent mixture including the solvent within a delivery device. The delivery device is configured to deliver a portion of the solvent or solvent mixture to a target residue to form a sample residue. The colorimetric dye is configured to undergo a chemical reaction when the sample residue contains a cannabinoid. The chemical reaction produces a visible color change that corresponds to a predetermined cannabinoid.

In an embodiment, the plurality of cannabinoids includes tetrahydrocannabinol (THC), cannabidiol (CBD), and cannabinol (CBN).

In an embodiment, the delivery device includes an absorbent material.

In an embodiment, the delivery device is a cotton swab that absorbs the solvent or solvent mixture, a pop or snap cotton swab that stores the solvent or solvent mixture in a shaft, or a wipe that absorbs the solvent or solvent mixture.

In an embodiment, storing the colorimetric dye includes applying the colorimetric dye to a solid support carrier and enclosing the solid support carrier within a first container, and storing the catalytic reagent includes absorbing, by the absorbent material the solvent mixture, and enclosing the absorbent material in a second container.

In an embodiment, the solid support carrier is a paper card, a paper sheet, a synthetic paper, or Whatman filter paper.

In an embodiment, storing the colorimetric dye includes affixing the colorimetric dye to the surface of the solid support carrier by a predetermined printing process, forming a reaction zone thereon.

In an embodiment, the predetermined printing process is one of letterpress, rotary gravure, rotary screen printing, flat screen printing, tampography, wax printing, contact dosing, ultrasonic sputter, flexographic, or spray or drop on demand printing.

In an embodiment, the printing process includes printing a liquid including the colorimetric dye on the surface of the solid support carrier by the predetermined printing process. The printing process further includes drying the printed solid support carrier. The printing process additionally includes cutting the solid support carrier into a predetermined shape.

In an embodiment, the liquid including the colorimetric dye is a homogenized solution or suspension of the colorimetric dye and one or more gelling agents. The homogenized solution or suspension has a predetermined viscosity suitable for printing.

In an embodiment, storing the colorimetric dye includes preparing a saturated dye solution of the colorimetric dye and absorbing the dye solution into the solid support carrier.

In an embodiment, the solvent mixture further includes the colorimetric dye, the catalytic reagent, and the surfactant.

In an embodiment, the colorimetric dye is vanillin, the catalytic reagent is p-toluene sulfonic acid, and the surfactant is fumed silica.

In an embodiment, the composition of the dry mixture is: 77% w/w Vanillin, 18% w/w p-Toluene Sulfonic Acid, and 5% w/w fumed silica.

In an embodiment, the colorimetric dye, the catalytic reagent, and a surfactant are in the form of a dry mixture of powders of the colorimetric dye, the catalytic reagent, and the surfactant. The dry mixture and the absorbent material are each enclosed and separated from each other within respective containers.

In an embodiment, the delivery device is a non-absorbent container enclosing the solvent mixture including the colorimetric dye, the catalytic reagent, the surfactant, and the solvent.

In an embodiment, the non-absorbent container is a syringe, a spray can, a pump spray bottle, a breakable ampoule, a blister pack, or a dropper bottle.

In an embodiment, the method further includes storing a heating device. The heating device is configured to produce heat sufficient to heat the residue in the form of a plant residue to a temperature sufficient to catalyze conversion of cannabinoid species within the plant residue to psychoactive THC.

In an embodiment, the method further includes storing a heating device. The heating device is configured to heat the residue in the form of a plant residue to a temperature greater than or equal to 100° C. for about 10 to about 60 seconds.

In an embodiment, the colorimetric dye is configured to undergo chemical reaction with the at least one cannabinoid in the form of liquids, gels or solid powders that are pure or admixed with cutting agents.

In an embodiment, the aldehyde is selected from the group consisting of Vanillin, DMAB, Metaldehyde, Anisaldehyde, Hydroxybenzaldehyde, Cinnamaldehyde, Saliscylaldehyde, or Nitrobenzaldehyde.

In an embodiment, the catalytic reagent increases the rate of appearance of the visible color change resulting from reaction between the colorimetric dye and the one or more predetermined cannabinoids.

In an embodiment, the catalytic reagent is at least one of a mineral acid or an organic acid in solid or liquid form.

In an embodiment, the catalytic reagent is selected from the group consisting of oxalic acid, citric acid, sodium bisulfate, or p-toluenesulfonic acid.

In an embodiment, solvent is an alcohol.

In an embodiment, the solvent is selected from the group consisting of methanol, ethanol, isopropyl alcohol, butanol, or benzyl alcohol.

In an embodiment, the surfactant is selected from the group consisting of Anionic, Cationic, Zwitterionic, Non-ionic, C10-C20 Ethoxylates, Fatty acid esters, Amine oxides, Sulfoxides, Phosphine oxides, fumed silica, or plant-derived surfactants.

In an embodiment, the surfactant is Sodium Lauryl Sulfate.

In an embodiment, the visible color change includes:

a turquoise color formation when the residue contains tetrahydrocannabinol (THC);

a pink color formation when the residue contains cannabidiol (CBD) or cannabinol (CBN); and

no color change or a yellow color formation when the residue does not contain a cannabinoid.

In certain embodiments, a shade or lightness of the color produced by the color change can correspond to a concentration of a cannabinoid present in the sample residue.

### DESCRIPTION OF DRAWINGS

Other objectives of this document will appear in the description and claims, with reference being made to the accompanying drawings, which form part of the specification.

FIG. 1 illustrates a solid support structure in the form of a diagnostic test paper strip made in accordance with the disclosed embodiments; Part A is plain white 300 gsm paper card and Part B is printed powder dye and catalytic reagent.

FIG. 2 illustrates a solid support structure in the form of a diagnostic test paper strip made in accordance with the disclosed embodiments; Part C is dip dried cellulose based or synthetic paper with reagents dry adsorbed within a matrix of the paper.

FIG. 3 illustrates an absorbent material in the form of a cotton swab made in accordance with the disclosed embodiments; Part D is a plastic polypropylene shaft handle; Part E is a cotton swab head, pre-wetted with any combination of (i) Solvent, (ii) Surfactant, (iii) Dye and (iv) Catalytic reagent.

FIG. 4 illustrates an absorbent material in the form of a cotton tipped snap swab made in accordance with the disclosed embodiments; Part F is an etched shaft tip which snaps when twisted releasing solvent contained in hollow shaft, into swab tip. Part G is a hollow plastic shaft filled with any combination of (i) Solvent, (ii) Surfactant, (iii) Dye, and (iv) Catalytic reagent. Part H is a snapped handle. Part I is a pre-filled hollow shaft releases liquid formulation into the swab tip.

FIG. 5 illustrates an absorbent material in the form of a cotton tipped pop swab made in accordance with the disclosed embodiments; Part J is a flexible plastic hollow shaft pre-filled with any combination of (i) Solvent, (ii) Surfactant, (iii) Dye and (iv) Catalytic reagent. Part K is a squeezed and “popped” flexible shaft, releasing liquid formulation. Part L is the swab tip receiving a liquid formulation.

FIG. 6 illustrates a dropper bottle made in accordance with the disclosed embodiments, pre-filled with any combination of (i) Solvent, (ii) Surfactant, (iii) Dye and (iv) Catalytic reagent.

FIG. 7 illustrates a dropper bottle made in accordance with the disclosed embodiments, pre-filled with any combination of (i) Solvent, (ii) Surfactant, (iii) Dye and (iv) Catalytic reagent.

FIG. 8 illustrates a pump spray bottle made in accordance with the disclosed embodiments, pre-filled with any combination of (i) Solvent, (ii) Surfactant, (iii) Dye and (iv) Catalytic reagent.

FIG. 9 illustrates a spray can made in accordance with t the disclosed embodiments, pre-filled with any combination of (i) Solvent, (ii) Surfactant, (iii) Dye and (iv) Catalytic reagent.

FIG. 10 illustrates a sachet made in accordance with the disclosed embodiments, pre-filled with any combination of (i) Dye and (ii) Catalytic reagent.

FIG. 11 illustrates a blister pack made in accordance with the disclosed embodiments, pre-filled with any combination of (i) Dye and (ii) Catalytic reagent.

FIG. 12 illustrates a breakable ampoule made in accordance with the disclosed embodiments, pre-filled with any combination of (i) Solvent, (ii) Surfactant, (iii) Dye and (iv) Catalytic reagent. Part M is plastic or glass or flexible polymer ampoule pre-filled with formulation. Part N is a broken/snapped/squashed ampule. Part O is contained formulation released.

FIG. 13 illustrates an absorbent material in the form of a wipe made in accordance with the disclosed embodiments, pre-wetted with any combination of (i) Solvent, (ii) Surfactant, (iii) Dye and (iv) Catalytic reagent.

FIGS. 14-17 illustrate a heating device and kit including the heating device according to embodiments of the present disclosure.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Contrary to existing approaches for the presumptive identification of THC differentiating it from other cannabinoids species including but not limited to CBD and CBN. It has been discovered that selected solvents, dry reactive dyes and catalytic reagents can be successfully mixed and packaged in various combinations to provide a highly selective, cheap, mass producible, portable detection device for the presumptive identification of THC, and provide operator safety when using the device.

### Solid Support

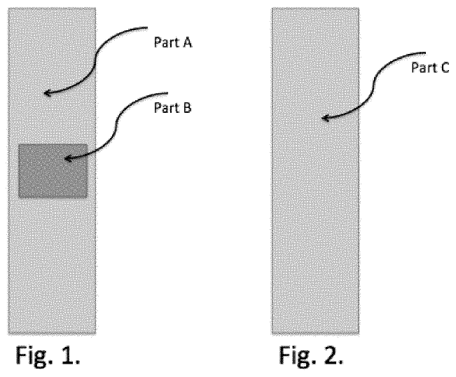

Suitable solid support carrier to which the dry colorimetric dyes, alone or in combination with catalytic reagents are adhered to, adsorbed to, or absorbed into, are dictated only by end use requirements. As an example, FIG. 1 illustrates a solid support structure made in accordance with the disclosed embodiments. Part A is a paper card (e.g., a plain white 300 gsm paper card) and Part B is a reaction zone formed by printing the colorimetric dye, alone or in combination with the catalytic reagent.

In accordance with embodiments of the present disclosure, and without limitation, the solid support substrate can be 300 gsm card paper, synthetic paper, Whatman filter paper, or similar.

FIG. 2 presents another embodiment of the solid support structure. Part C is dip dried cellulose based or synthetic paper with the colorimetric dye, alone or in combination with the catalytic reagent adsorbed within a matrix of the solid support structure.

### Absorbent Material

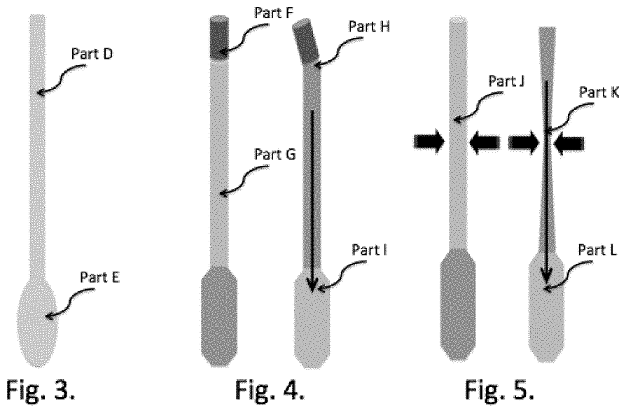

In accordance with embodiments of the present disclosure, and without limitation, in one embodiment, the absorbent material is in the form of a pre-wetted cotton swab, as illustrated in FIG. 3. The cotton swab includes a handle (Part D) and a tip or head (Part E). The pre-wetting can be achieved by simple dip and/or rapid immersion of the cotton swab matrix into large volume pre-mixed solvent or solvent mixture, vat or micro-jet spray or similar. Pre-wetting is a fully automated process utilizing conventional conveyor, hopper, spray machinery.

In another embodiment and without limitation the absorbent material is in the form of a “snap cotton swab,” as illustrated in FIG. 4. The hollow swab handle (Part G) is pre-filled in fully automated commercial fill processes with any combination of (i) Solvent, (ii) Surfactant, (iii) Dye and (iv) Catalytic reagent, as described herein. A tip of the swab handle (Part F) has etched groove applied during swab manufacture which is easily snapped (Part H) between thumb and forefinger, releasing said shaft contents down and into the cotton tip (Part I).

In yet another embodiment and without limitation the absorbent material is in the form of a “pop cotton swab,” as illustrated in FIG. 5. A hollow flexible swab handle (Part J) is pre-filled in fully automated commercial fill processes with any combination of (i) Solvent, (ii) Surfactant, (iii) Dye and (iv) Catalytic reagent, as described in preceding sections of this disclosure. The handle of the swab handle is easily squeezed (Part K) and small closure contained with hollow shaft handle broken or “popped” between thumb and forefinger, releasing said shaft contents down and into the cotton tip (Part L).

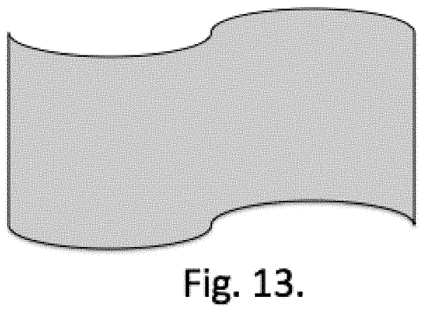

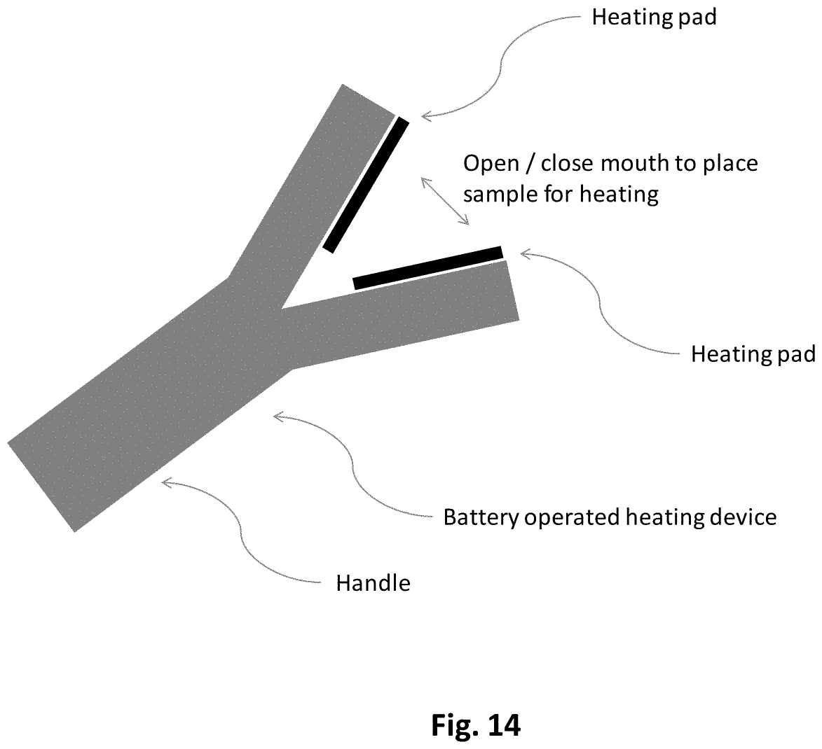

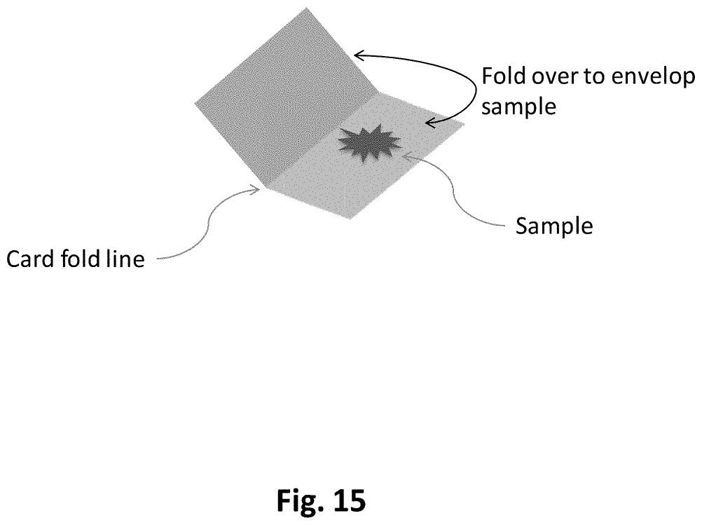

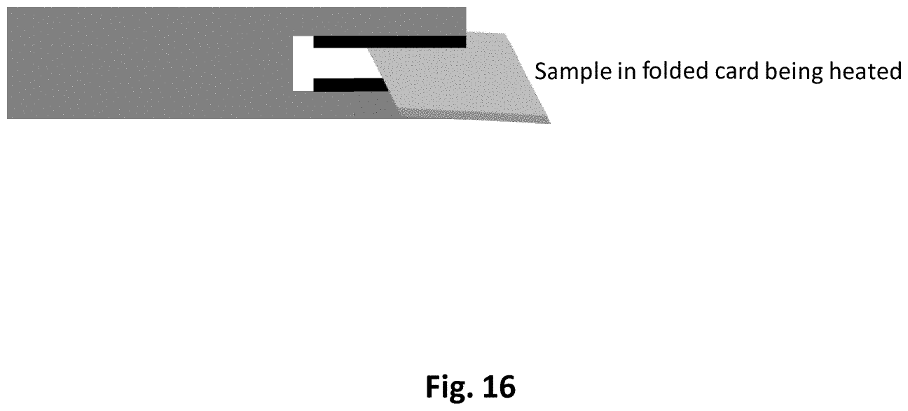

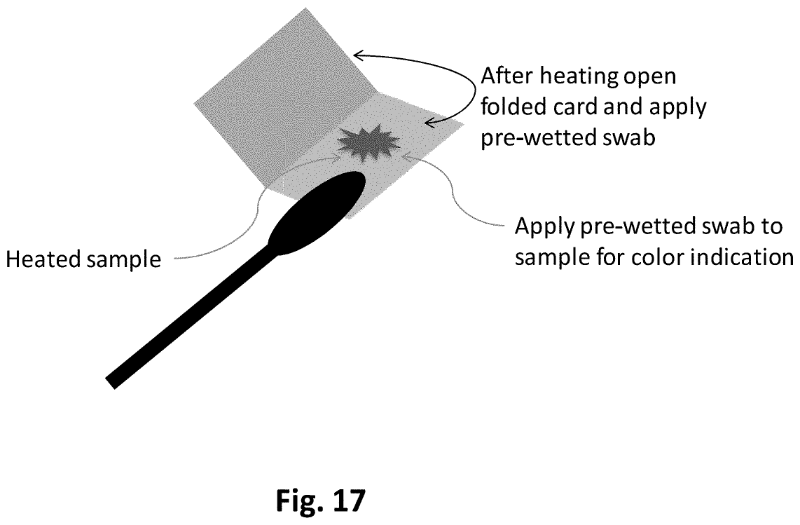

In yet another embodiment and without limitation the absorbent material is in the form of a “wipe,” as illustrated in FIG. 13. The wipe can be made of any natural or synthetic polymeric fibers and pre-wetted with any combination of (i) Solvent, (ii) Surfactant, (iii) Dye and (iv) Catalytic reagent, as described in preceding sections of this disclosure. The “wipe” is simply applied to the suspect residues and pressed into or wiped across said residue for collection and presumptive identification of said residues.

### Containers

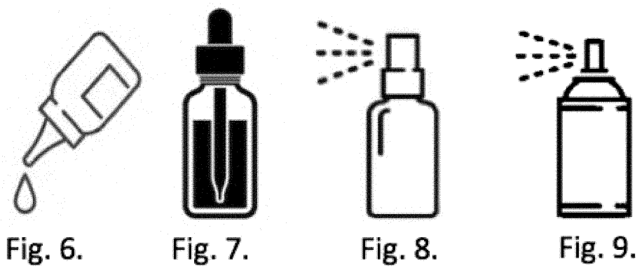

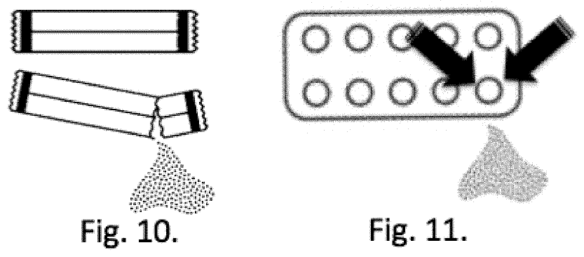

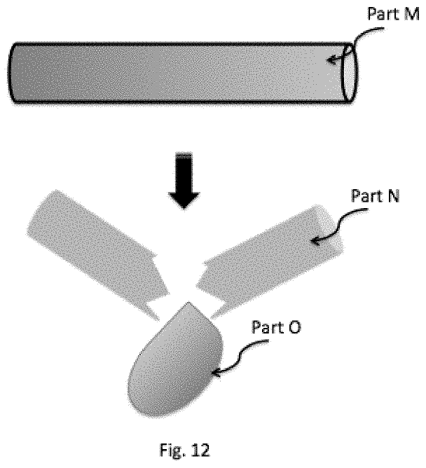

An ampoule (FIG. 12), blister pack (FIG. 11), dropper bottle (FIGS. 6-7), pump spray bottle (FIG. 8), a spray can (FIG. 9) may be used to contain any combination of (i) Solvent, (ii) Surfactant, (iii) Dye and (iv) Catalytic reagent, as described in preceding sections of this disclosure. A sachet suitable for receipt of at least the colorimetric dye, alone or in combination with the catalytic reagent, is illustrated in FIG. 10.

As further illustrated in FIG. 12, the breakable ampule can be formed from plastic or glass or flexible polymer pre-filled with formulation (Part M). Part N is a broken/snapped/squashed ampule. Part 0 is contained formulation released.

All of the above examples are effective methods to safely store any formulation in any combination of (i) Solvent, (ii) Surfactant, (iii) Dye and (iv) Catalytic reagent, as described in preceding sections of this disclosure. Allowing mass production, packaging, shipping and transport and carriage by end user prior to use.

### Presumptive Colorimetric Dye

The presumptive colorimetric dye, also referred to as a colorimetric reagent, can be configured to produce a known visual color indication in the presence of THC and other cannabinoids (e.g., CBD, CBN, etc.) That is, the color indication is unique to a specific cannabinoid. So configured, the kit can identify and differentiate THC from other cannabinoids within unknown suspect residues, be they solid or liquid.

In accordance with embodiments of the present disclosure, and without limitation, in one embodiment, colorimetric dyes for the presumptive identification of THC can include, but are not limited to, Vanillin, DMAB, Metaldehyde, Anisaldehyde, Hydroxybenzaldehyde, Cinnamaldehyde, Saliscylaldehyde, Nitrobenzaldehyde. An exemplary aldehyde is Vanillin.

### Solvent

In accordance with embodiments of the present disclosure, and without limitation, suitable solvents can be alcohols. Exemplary alcohols can include, but are not limited to, methanol, ethanol, isopropyl alcohol, butanol, and/or benzyl alcohol.

### Surfactant

In accordance with embodiments of the present disclosure, and without limitation, suitable surfactants can include, but are not limited to, C10-C20 ethoxylated non-ionic surfactants.

### Catalytic Reagent

In accordance with embodiments of the present disclosure, and without limitation, in one embodiment, the catalytic reagent is as an acid. Examples of suitable acids can include, but are not limited to, mineral acids or organic acids, solid or liquid. An exemplary acid is p-toluenesulfonic acid.

Pre-wetting absorbent material

In certain embodiments, any combination of the solvent, the surfactant, the catalytic reagent, and the solvent are mixed as previously described. A dry absorbent material (e.g., a dry cotton swab or wipe) is immersed into the solution and then packaged in suitable form fill seal sachet (see below).

### Packaging

In accordance with the present disclosure, and without limitation, in one embodiment of the kit, the pre-wetted absorbent material for sample collection can be packaged in moisture and UV resistant packages prior to use. In an embodiment, the packaging can be a tare open, form, fill and seal sachet. The sachet can be constructed from commercially available Paper/PET 12 μm/AL 7 μm/PE 50 product, which is an extremely cheap, mass produced material. The dry solid support carrier (e.g., a mineral paper strip), can be packaged individually in separate paper based sachets. All sachets are formed by vertical and/or horizontal form/seal machines, which are well known in the art.

### Use of Kit

In accordance with the present disclosure, and without limitation, in one embodiment, the kit is carried (e.g., in a pocket, belt case, glove box, brief case, etc.) When a suspect residue is observed, both the pre-wetted absorbent material and solid support carrier are removed from respective sachet packaging. The absorbent material is rubbed into the suspect residue, liquid, gel, solid and/or across suitable surfaces for several seconds, to facilitate the collection of a representative sample of the suspect residue. To complete the process, the absorbent material is transferred to the solid support carrier, which contains the printed or pre-adsorbed colorimetric reagent. The transfer can include pressing or dabbing the absorbent material containing the representative sample onto the solid support structure. This facilitates full mixing of all components and enhances any presumptive colorimetric indication for THC or other cannabinoids, with different color indications for both THC and other cannabinoids.

In yet another embodiment and in accordance with the present disclosure, and without limitation, with the kit is provided a heating device. The heating device can be capable of heating a suspect residue (e.g., a plant residue) to a temperature sufficient to catalyze conversion of a cannabinoid species within the suspect residue (e.g., to psychoactive THC). In an embodiment, the heating device can be configured to heat the suspect residue to a temperature greater than 100° C. (e.g., within the range between about 100° C. to about 150° C. for a time duration between about 10 seconds to about 60 seconds.

The heating device can include a power source or be configured to receive power from an external source. As an example, the heating device can be in the form of a battery powered miniaturized flat, dual sided, plate heating structure. A piece of suspect plant material can be placed therebetween and heated to catalyze conversion of cannabinoid precursors to psychoactive THC. Total THC content within the suspect plant residue can then be analyzed to differentiate Hemp from Marijuana. After about 10 seconds to about 60 seconds of heating plant material at or above 100° C., conversion of cannabinoid precursors to psychoactive THC is complete. At this time both the pre-wetted swab and solid support carrier are removed from respective sachet packaging. The cotton swab is rubbed into the heat converted suspect plant residue for several seconds to facilitating full mixing of all components and enhancing any presumptive colorimetric indication for THC or other cannabinoids, with different color indications for both THC and other cannabinoids

### Color Indications

In accordance with the embodiments of the present disclosure, and without limitation, example results from using the kit disclosed herein are provided: (Control—Negative) no color change—White, (Positive THC Indication) Color Rapid change from White to pink—red (THC<0.5%), purple (THC<5%), dark blue (YHC<10%) Turquoise/Blue (THC>10%), (CBD Indication) No color change white (time for observation<60sec), (CBN Indication) No color change white (time for observation<60sec). That is, in addition to color change for identification of the presence of a cannabinoid in the sample residue, a shade or lightness of the color produced by the color change can correspond to a concentration of the cannabinoid present in the sample residue.

### EXAMPLES

The following examples illustrate certain specific embodiments of the disclosure and are not meant to limit the scope of the disclosed embodiments.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

In accordance with the embodiments of the present disclosure, and without limitation, a presumptive kit for the detection and identification of THC, differentiating it from CBN and CBD is produced by pre-wetting a cotton swab with 0.01 to 0.05 mL of a saturated solution of solvent and catalytic reagent. A paper strip (e.g., the solid support carrier) having the colorimetric reagent affixed thereon is made by producing a saturated solution of colorimetric reagent in isopropyl alcohol and dip drying a sheet of Whatman No. 1 filter paper into said solution, and cutting dried sheet into strips and packaging said strips. The pre-wetted absorbent material and pre-absorbed strip are packaged individually into hermetically form fill sealed Paper/PET12 μm/AL7 μm/PE50 sachet.

### Example 2

In yet another embodiment, in accordance with the present disclosure, and without limitation, a presumptive kit for the detection and identification of THC, differentiating it from CBN and CBD is produced by pre-wetting a cotton swab with an homogenized solution of 17.4 wt. % Vanillin, 17.4 wt. % p-toluenesulfonic acid, dissolved in 65.2 wt. % methanol solvent. The pre-wetted swab is individually packaged into hermetically form fill sealed Paper/PET12 μm/AL7 μm/PE50 sachet.

### Example 3

In yet another embodiment, in accordance with the present disclosure, and without limitation, a presumptive kit for the detection and identification of THC, differentiating it from CBN and CBD is produced by adding homogenized dry powder mix of Vanillin (77% w/w), p-Toluene Sulfonic Acid (18% w/w), and fumed silica (5% w/w) into form fill seal sachets. A pre-wetted swab with benzyl alcohol, butanol, or isopropyl alcohol is individually packaged into hermetically form fill sealed Paper/PET12 um/AL7 um/PE50 sachet.

While the test kit has been shown and described in detail, it is obvious that the disclosed embodiments are not to be considered as limited to the exact form disclosed, and that changes in detail and construction may be made therein within the scope of the disclosed embodiments without departing from the spirit thereof.

The abbreviations used herein have their conventional meanings within the chemical and biological arts.

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the disclosed embodiments. It should be understood that various alternatives to the embodiments described herein may be employed.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this the disclosed embodiments. The following definitions are provided to facilitate understanding of certain terms used frequently herein and are not meant to limit the scope of the present disclosure.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosed embodiments.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the disclosed embodiments. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

### REFERENCES

The following references are incorporated by reference in their entirety.

### Patent Documents

EP 0991984(A1)—2000 Apr. 12—Programming Apparatus
WO 9954739(A1) 1999 Oct. 28—Detection and determination of phenol-like substances by means of direct wet-chemical methods.
US 2017/0234897A1—Methods for cannabinoid quantification.
U.S. Pat. No. 4,816,415A—Cannabinoid detection method.
U.S. Pat. No. 3,715,189A—Qualitative analysis device
US 2015/0017732A1—Colorimetric method to detect illicit drugs
US 2007/0077660A1—Method for the detection of a cannabinoid, detection kit, and developing solvent.
U.S. Pat. No. 4,771,005A—Reagents, test kits and methods for the detection of cannabinoids.
EP 1825262B1—Delta-9-tetrahydrocannabinol detection.
U.S. Pat. No. 5,817,766A—Reagents for a cannabinoid immunoassay.
JP 2005-506520A—New cannabinoid detection.
US 2019/0185946A1—Methods and kits for classifying cannabinoid production in cannabis plants.
WO 1988/009496A1—Cannabinoid detection system.
U.S. Pat. No. 9,726,684B1—Compositions for target substance detection and measurement.

### Non-Patent Literature

[1] Nahas, G. G. (1973). From Bhang to delta-9-THC: four thousand years of history. Marihuana Deceptive Weed. Raven, New York, pp. 1-58.
[2] https://wdr.unodc.org/wdr2019/
[3]https://en.wikipedia.org/wiki/Cannabinoid
[4] https://www.gimitec.com//file/720005812en.pdf
[5] UNODC—laboratory and scientific section united nations office on drugs and crime, vienna, recommended methods for the identification and analysis of cannabis and cannabis products (revised and updated) manual for use by national drug analysis laboratories united nations new york, 2009.
[6] https://www.who.int/medicines/access/controlled-substances/IsomersTHC.pdf
[7] El-Darawy, Z. I. and Mobarak, Z. m. Studies on hashish IV. Color reactions of Cannabinols. Qualitas Plantarum et Materiae Vegetables. December 1972, Vol. 22. pp 7-131.
[9] https://doi.org/10.1159/000489287
[10] Kelly, J. F., et. al. The Non-Specificity of the Duquenois-Levine Field Test for Marijuana. The Open Forensic Science Journal, 2012, 5, 4-8.
[11] Blackie, J. W. New Color Reactions for Cannabis sativa Resin. Ind. Eng. Chem. Anal. Ed, 1941, 13 (2), pp 96-97.
[12] Kelly, J. False positives equal false justice. 2008
[13] ASAHINA, H. UNODC—STUDIES ON CANNABIS OBTAINED FROM HEMP PLANTS GROWN IN JAPAN, 1957. PP 17-20.
[14] DE FAUBERT MAUNDER, M. J. UNODC—TWO SIMPLE COLOUR TESTS FOR CANNABIS. 1969. PP 37-42.
[15] Commercial TLC Unit—https://www.alpha-cat.org

1. A portable detection kit for identifying the presence of cannabinoids, comprising:
a colorimetric dye,
a catalytic reagent,
a solvent,
a surfactant, and
a delivery device containing a solvent or solvent mixture including the solvent;
wherein the delivery device is configured to deliver a portion of the solvent or solvent mixture to a target residue to form a sample residue;
wherein the colorimetric dye undergoes a chemical reaction when the sample residue contains a cannabinoid; and
wherein the chemical reaction produces a visible color change that corresponds to a predetermined cannabinoid of a plurality of cannabinoids.

2. The portable detection kit as in claim 1, wherein the plurality of cannabinoids includes one or more of tetrahydrocannabinol (THC), cannabidiol (CBD), or cannabinol (CBN).

3. The portable detection kit of claim 1, wherein the delivery device includes an absorbent material.

4. The portable detection kit of claim 3, wherein the delivery device is a cotton swab that absorbs the solvent or solvent mixture, a pop or snap cotton swab that stores the solvent or solvent mixture in a shaft, or a wipe that absorbs the solvent or solvent mixture.

5. The portable detection kit as in claim 3, wherein the colorimetric dye is received by a solid support carrier and the absorbent material contains the solvent mixture including the catalytic reagent and the surfactant, and wherein the solid support carrier and the absorbent material are each enclosed and separated from each other in respective containers.

6. The portable detection kit as in claim 5, wherein the solid support carrier is a paper card, a paper sheet, a synthetic paper, or Whatman filter paper.

7. The portable detection kit as in claim 3, wherein the solvent mixture further comprises the colorimetric dye, the catalytic reagent, and the surfactant.

8. The portable detection kit of claim 7, wherein the colorimetric dye is vanillin, the catalytic reagent is p-toluene sulfonic acid, and the surfactant is fumed silica.

9. The portable detection kit of claim 8, wherein the composition of the dry mixture is:
77% w/w Vanillin;
18% w/w p-Toluene Sulfonic Acid; and
5% w/w fumed silica.

10. The portable detection kit as in claim 3, wherein the colorimetric dye, the catalytic reagent, and the surfactant are in the form of a dry mixture of powders of the colorimetric dye, the catalytic reagent, and the surfactant, and wherein the dry mixture and the absorbent material are each enclosed and separated from each other within respective containers.

11. The portable detection kit of claim 1, wherein the delivery device is a non-absorbent container enclosing a solvent mixture including the colorimetric dye, the catalytic reagent, the surfactant, and the solvent.

12. The portable detection kit of claim 11, wherein the non-absorbent container is a syringe, a spray can, a pump spray bottle, a breakable ampoule, a blister pack, or a dropper bottle.

13. The portable detection kit as in claim 5, further comprising a heating device configured to produce heat sufficient to heat the residue in the form of a plant residue to a temperature greater than or equal to 100° C. for about 10 to about 60 seconds, thereby catalyzing conversion of cannabinoid species within the plant residue to psychoactive THC.

14. The portable detection kit as in claim 1, wherein the colorimetric dye is configured to undergo chemical reaction with the at least one cannabinoid in the form of liquids, gels or solid powders that are pure or admixed with cutting agents.

15. The portable detection kit as in claim 1, wherein the colorimetric dye is an aldehyde.

16. The portable detection kit as in claim 15, wherein the aldehyde is selected from the group consisting of Vanillin, DMAB, Metaldehyde, Anisaldehyde, Hydroxybenzaldehyde, Cinnamaldehyde, Saliscylaldehyde, or Nitrobenzaldehyde.

17. The portable detection kit as in claim 1, wherein the catalytic reagent increases the rate of appearance of the visible color change resulting from reaction between the colorimetric dye and the one or more predetermined cannabinoids.

18. The portable detection kit as in claim 1, wherein the catalytic reagent is at least one of a mineral acid or an organic acid in solid or liquid form.

19. The portable detection kit as in claim 18, wherein the catalytic reagent is selected from the group consisting of oxalic acid, citric acid, sodium bisulfate, or p-toluenesulfonic acid.

20. The portable detection kit as in claim 1, wherein solvent is an alcohol.

21. The portable detection kit of claim 20, wherein the solvent is selected from the group consisting of methanol, ethanol, isopropyl alcohol, butanol, or benzyl alcohol.

22. The portable detection kit as in claim 1, wherein the surfactant is selected from the group consisting of Anionic, Cationic, Zwitterionic, Non-ionic, C10-C20 Ethoxylates, Fatty acid esters, Amine oxides, Sulfoxides, Phosphine oxides, fumed silica, or plant-derived surfactants.

23. The portable detection kit as in claim 1, wherein the surfactant is Sodium Lauryl Sulfate.

24. The portable detection kit as in claim 1, wherein the visible color change comprises:
a turquoise color formation when the residue contains tetrahydrocannabinol (THC);
a pink color formation when the residue contains cannabidiol (CBD) or cannabinol (CBN); and
no color change or a yellow color formation when the residue does not contain a cannabinoid.

25. A method of preparing a portable detection kit for detecting the presence of cannabinoids, comprising:
storing a colorimetric dye;
storing a catalytic reagent;
storing a surfactant; and
storing a solvent or solvent mixture including the solvent within a delivery device;
wherein the delivery device is configured to deliver a portion of the solvent or solvent mixture to a target residue to form a sample residue;
wherein the colorimetric dye undergoes a chemical reaction when the sample residue contains a cannabinoid; and

[symbol]

26. [symbol] wherein the chemical reaction produces a visible color change that corresponds to a predetermined cannabinoid. The method according to claim 25, wherein the plurality of cannabinoids includes tetrahydrocannabinol (THC), cannabidiol (CBD), and cannabinol (CBN).

27. The method according to claim 25, wherein the delivery device includes an absorbent material.

28. The method according to claim 27, wherein the delivery device is a cotton swab that absorbs the solvent or solvent mixture, a pop or snap cotton swab that stores the solvent or solvent mixture in a shaft, or a wipe that absorbs the solvent or solvent mixture.

29. The method as in claim 27, wherein storing the colorimetric dye comprises applying the colorimetric dye to a solid support carrier and enclosing the solid support carrier within a first container, and wherein storing the catalytic reagent comprises absorbing, by the absorbent material the solvent mixture, and enclosing the absorbent material in a second container.

30. The method as in claim 29, wherein the solid support carrier is a paper card, a paper sheet, a synthetic paper, or Whatman filter paper.

31. The method according to claim 29, wherein storing the colorimetric dye comprises affixing the colorimetric dye to the surface of the solid support carrier by a predetermined printing process, forming a reaction zone thereon.

32. The method according to claim 31, wherein the predetermined printing process is one of letterpress, rotary gravure, rotary screen printing, flat screen printing, tampography, wax printing, contact dosing, ultrasonic sputter, flexographic, or spray or drop on demand printing.

33. The method according to claim 31, wherein the printing process comprises:
printing a liquid including the colorimetric dye on the surface of the solid support carrier by the predetermined printing process;
drying the printed solid support carrier; and
cutting the solid support carrier into a predetermined shape.

34. The method according to claim 33, wherein the liquid including the colorimetric dye is a homogenized solution or suspension of the colorimetric dye and one or more gelling agents, and wherein the homogenized solution or suspension has a predetermined viscosity suitable for printing.

35. The method according to claim 29, wherein storing the colorimetric dye comprises:
preparing a saturated dye solution of the colorimetric dye; and
absorbing the dye solution into the solid support carrier.

36. The method according to claim 27, wherein the solvent mixture further comprises the colorimetric dye, the catalytic reagent, and the surfactant.

37. The method according to claim 36, wherein the colorimetric dye is vanillin, the catalytic reagent is p-toluene sulfonic acid, and the surfactant is fumed silica.

38. The method according to claim 37, wherein the composition of the dry mixture is:
77% w/w Vanillin;
18% w/w p-Toluene Sulfonic Acid; and
5% w/w fumed silica.

39. The method according to claim 27, wherein the colorimetric dye, the catalytic reagent, and a surfactant are in the form of a dry mixture of powders of the colorimetric dye, the catalytic reagent, and the surfactant, and wherein the dry mixture and the absorbent material are each enclosed and separated from each other within respective containers.

40. The method according to claim 25, wherein the delivery device is a non-absorbent container enclosing the solvent mixture including the colorimetric dye, the catalytic reagent, the surfactant, and the solvent.

41. The method according to claim 40, wherein the non-absorbent container is a syringe, a spray can, a pump spray bottle, a breakable ampoule, a blister pack, or a dropper bottle.

42. The method according to claim 29, further comprising storing a heating device, the heating device being configured to produce heat sufficient to heat the residue in the form of a plant residue to a temperature sufficient to catalyze conversion of cannabinoid species within the plant residue to psychoactive THC.

43. The method according to claim 29, further comprising storing a heating device, wherein the heating device is configured to heat the residue in the form of a plant residue to a temperature greater than or equal to 100° C. for about 10 to about 60 seconds.

44. The method according to claim 25, wherein the colorimetric dye is configured to undergo chemical reaction with the at least one cannabinoid in the form of liquids, gels or solid powders that are pure or admixed with cutting agents.

45. The method according to claim 25, wherein the colorimetric dye is an aldehyde.

46. The method according to claim 45, wherein the aldehyde is selected from the group consisting of Vanillin, DMAB, Metaldehyde, Anisaldehyde, Hydroxybenzaldehyde, Cinnamaldehyde, Saliscylaldehyde, or Nitrobenzaldehyde.

47. The method according to claim 25, wherein the catalytic reagent increases the rate of appearance of the visible color change resulting from reaction between the colorimetric dye and the one or more predetermined cannabinoids.

48. The method according to claim 25, wherein the catalytic reagent is at least one of a mineral acid or an organic acid in solid or liquid form.

49. The method according to claim 48, wherein the catalytic reagent is selected from the group consisting of oxalic acid, citric acid, sodium bisulfate, or p-toluenesulfonic acid.

50. The method according to claim 25, wherein solvent is an alcohol.

51. The method according to claim 50, wherein the solvent is selected from the group consisting of methanol, ethanol, isopropyl alcohol, butanol, or benzyl alcohol.

52. The method according to claim 25, wherein the surfactant is selected from the group consisting of Anionic, Cationic, Zwitterionic, Non-ionic, C10-C20 Ethoxylates, Fatty acid esters, Amine oxides, Sulfoxides, Phosphine oxides, fumed silica, or plant-derived surfactants.

53. The method according to claim 25, wherein the surfactant is Sodium Lauryl Sulfate.

54. The method according to claim 25, wherein the visible color change comprises:
a turquoise color formation when the residue contains tetrahydrocannabinol (THC);
a pink color formation when the residue contains cannabidiol (CBD) or cannabinol (CBN); and
no color change or a yellow color formation when the residue does not contain a cannabinoid.
